(19) United States
(12) Patent Application Publication
(10) Pub. No.: US 2004/0137032 A1
(43) Pub. Date: Jul. 15, 2004

(54) Combinations of calcium phosphates, bone growth factors, and pore-forming additives as osteoconductive and osteoinductive composite bone grafts

(76) Inventor: Francis W. Wang, Montgomery Village, MD (US)

Correspondence Address: STEVENS, DAVIS, MILLER & MOSHER, LLP, Suite 850, 1615 L Street NW, Washington, DC, 20036, US

(21) Appl. No.: 10/384,702

(22) Filed: Mar. 11, 2003

Related U.S. Application Data
(60) Provisional application No. 60/365,141, filed on Mar. 15, 2002.

(51) Int. Cl. A61K 9/14

(52) U.S. Cl. 424/423

(57) ABSTRACT

The first element of the invention is a moldable, resorbable composite bone graft consisting of calcium phosphate cement, an aqueous medium, and biodegradable polymer microspheres or other porogens. The composite bone graft is osteoconductive, i.e., the degradation of the microspheres or other porogens leads to macropores that facilitate the growth of osteoblasts into the bone grafts, and the degradation rate of microspheres or other porogens can be controlled such that the formation of macrpores is in tune with the ingrowth of osteoblasts. Additionally, growth factors or other bioactive agents can be incorporated into the moldable, resorbable composite bone grafts. The growth factors can be added to the liquid component of the bone grafts or, can be encapsulated in a biodegradable porogen and then added to the solid component of the bone grafts (or both). The release patterns of the growth factors are modulated by varying the volume fraction and the dissolution rate of the pore-forming particulates, which are biodegradable polymer microspheres or other watersoluble particles.

This application claims priority from of U.S. Provisional Application No. 60/365,141, which was filed on Mar. 15, 2002, herein incorporated by reference in its entirety.

### BACKGROUND OF THE INVENTION

1. Field of the Invention

There exists in the orthopedic market a moldable, self-setting, calcium phosphate cement (CPC) comprising an equimolar mixture of tetracalcium phosphate (TTCP) powder and dicalcium phosphate anhydrous (DCPA) powder. The CPC powder is mixed with water or an aqueous solution to form a workable paste that can be shaped to fit the contour of a wound. The cement paste sets in approximately 20 to 30 minutes and precipitates as microcrystalline hydroxyapatite that can be resorbed by the body and replaced with new bone. The calcium phosphate cement is biocompatible and osteoconductive. However, this osteoconductivity is usually observed only in restricted areas adjacent to the pre-existing host bone. Furthermore, the cement does not exhibit osteoinductivity, one of the desirable characteristics of a bone graft.

2. Background

Calcium phosphate ceramics meet some of the requirements for bone grafts that have been suggested by the National Institutes of Health. Such ceramics are biocompatible and exhibit osteoconductivity. However, this osteoconductivity is usually observed in restricted areas adjacent to the pre-existing host bone. Some studies have reported that bone marrow cells initiate osteogenesis in porous calcium phosphate ceramics that have been implanted into subcutaneous, intramuscular, or orthotopic sites. Although these biocompatible, porous bone grafts meet some of the aforementioned requirements, they may be further improved by making them injectable or moldable, and by providing controlled release of osteoinductive proteins or oligopeptides, such as adhesive factors, growth factors or other agents.

Other scientists made important advances toward the improvement of porous calcium-phosphate bone grafts. They have developed a moldable, self-setting calcium phosphate cement (CPC). The cement powder is mixed with water to form a workable paste that can be shaped to fit the contours of a wound. The cement precipitates as microporous, microcrystalline hydroxyapatite (HAP) that is gradually resorbed by the body and replaced with new bone. The cement sets in minutes and extensive testing has found the cement to be completely biocompatible. The mechanical strength of the CPC (typically 10.8 MPa in diametral tensile strength and 66.1 MPa in compressive strength) is not suitable for load-bearing applications, but can still be used clinically to treat craniofacial defects.

Therefore, the purpose of this invention is to provide novel, biocompatible and resorbable, composite bone grafts by improving calcium phosphate cements. Each of the moldable composite bone grafts, which can be shaped (before or during surgery) to fit the contour of a wound, has been formulated to have the following characteristics: It is osteoinductive, i.e., it serves as a carrier to release, at controllable rates, one or more growth factors, or cells (osteoblasts or transfected cells) or vectors for gene transfer; and it is osteoconductive, i.e., it allows bone cells to grow into it.

### SUMMARY OF THE INVENTION

In order to enhance the rate of CPC resorption and bony ingrowth into the implant, investigators have created macropores in the CPC by, for example, a particulate leaching technique using mannitol crystals as porogens. However, when the mannitol crystals are included at a high volume fraction (approx. 0.6), the grafts become too weak to adequately maintain their shape for use in vivo.

To remedy this reduction in strength, biodegradable poly(lactide-co-glycolide) (PLGA) microspheres between 170 μm and 360 μm in diameter are used to impart macroporosity to the cement. When implanted, the polymer microspheres degrade to form macropores for bony ingrowth. The strengthening of the graft from bony ingrowth and the deposition of new bone offsets the weakening of the graft due to polymer degradation. In addition, PLGA is used clinically in suture materials and is considered to be non-toxic and biocompatible both in vitro and in vivo.

Calcium phosphate ceramics are used that have been implanted into subcutaneous, intramuscular, or orthotopic sites. Although these biocompatible, porous bone grafts meet some of the aforementioned requirements, they may be further improved by making them injectable or moldable, and by providing controlled release of osteoinductive proteins or oligopeptides, such as adhesive factors, growth factors or other agents

### BRIEF DESCRIPTION OF THE DRAWINGS

FIG. 1. The excitation and the emission spectra of the rubrene reference solution at the emission wavelength of 670 nm and the excitation wavelength of 475 nm.

FIG. 2. The SEM (scanning electron microscopy) photomicrograph of the fractionated PLGA microspheres.

FIG. 3. The SEM (scanning electron microscopy) photomicrograph of the fractionated PLA microspheres.

FIG. 4. The x-ray diffraction pattern of a PLGA/CPC composite.

FIG. 5. The average fractional decrease in mass of the PLGA/CPC composite as a function of the time of immersion in a saliva-like solution (n=3).

FIG. 6. The SEM photomicrograph of the PLGA/CPC composite after the dissolution of PLGA microspheres in a saliva-like solution.

FIG. 7. The average diametral tensile strength of the PLGA/CPC composite as a function of the time of immersion in a saliva-like solution (n=5).

FIG. 8. The fluorescence intensity of Protein A, released from the PLGA/CPC composite, as a function of the time of immersion in a saliva-like solution. The upper plot and the lower plot are for the composites containing 100% and 35%, respectively, of Protein A-encapsulating microspheres.

FIG. 9. The fluorescence intensity of Protein A, released from the PLGA/CPC composite, as a function of the time of immersion in a saliva-like solution. The filled circles and the filled squares are for the composites containing 100% and 35%, respectively, of Protein A-encapsulating microspheres.

FIG. 10. The average fluorescence intensity of Protein A released from the PLA/CPC composite as a function of the time of immersion in a saliva-like solution (n=4).

FIG. 11. The fluorescence intensity of Protein A, released from the mannitol/CPC composite, as a function of the time of immersion in a saliva-like solution. The filled circles, the open circles and the filled triangles are for the composites containing 0%, 35%, and 60% by volume of mannitol crystals.

### DETAILED DESCRIPTION OF THE INVENTION

A. Composite Bone Grafts Including of Calcium Phosphate Cement and Biodegradable Polymer Microspheres

Since the development in the first CPC consisting of TTCP and DCPA many different combinations of calcium- and phosphate-containing compounds have been investigated as potential CPC materials. While the compositions of the invention may specify, for the sake of clarity, the CPC consisting of TTCP and DCPA as a component, it is worth noting that the invention does not exclude the use of other calcium phosphate cements as the component.

In order to increase the resorption of a CPC bone graft and to enhance the growth of new bone into the graft, macropores may be created in the CPC graft by a particulate leaching technique using biodegradable polymer microspheres as pore-forming particles.

In a typical example, the powder component of a composite bone graft is made from the CPC powder [an equimolar mixture of tetracalcium phosphate (TTCP) powder and dicalcium phosphate anhydrous (DCPA) powder] and biodegradable microspheres of a poly(lactide-co-glycolide) copolymer (PLGA, 50:50 comonomer molar ratio). The volume fraction and the diameter of the microspheres were approximately 0.6 and approximately 170 to 360 μm, respectively.

Each specimen of the PLGA/CPC composite bone graft was prepared by mixing 0.25 g of the powder component of the composite bone graft with 0.062 g of water (as the liquid component of the graft) to make a paste. The paste was placed into a stainless steel mold, 6.4 mm in diameter and 4.4 mm in height. Two 10 mm long steel inserts were placed sandwiching the specimen, and the mold was placed in a 37° C. humidity chamber. A load of 20 N was applied to the specimen for 24 h. The specimen was then removed and used for the measurements of the diametral tensile strength (DTS) and the mass loss during the immersion of the specimen at a 37° C. in a saliva-like solution.

The mass loss due to the degradation and the dissolution of PLGA microspheres was complete in seven weeks. The initial value of 6.4 MPa for the diametral tensile strength of the PLGA/CPC composite bone graft (containing 60% by volume of PLGA microspheres) was nearly twice the value of 3.5 MPa for a similar CPC bone graft that contained only 29% by volume of mannitol crystals instead of PLGA microspheres as pore-forming particulates.

When the PLGA/CPC composite bone graft is implanted, the polymer microspheres initially stabilize the graft but gradually degrade, leaving behind pores of appropriate sizes for colonization by osteoblasts. The strengthening of the graft from bony ingrowth and the deposition of new bone should offset the weakening of the graft due to polymer degradation, since the degradation rate can be controlled such that the formation of macropores is in tune with the ingrowth of new bone. In addition, PLGA is used clinically as a suture material and is considered to be non-toxic and biocompatible both in vitro and in vivo. Thus, the PLGA/CPC composite bone graft is osteoconductive because the degradation of microspheres leads to macropores that facilitate the growth of osteoblasts into the bone graft.

Biodegradable polymer microspheres can be made from one of the biodegradable and biocompatible polymers that include poly(L-lactide), poly(glycolide), poly(D,L-lactide), poly(ε-caprolactone), poly(carbonates) and poly(orthoesters), as well as poly(anhydrides), poly(D,L-lactide-co-L-lactides), poly(D,L-lactide-co-glycolides) and poly(L-lactide-co-glycolides), of various comonomer molar ratios. The degradation rate of the polymer microspheres can be controlled through the selection of a polymer with an appropriate degradation rate, and through the addition of an acidic or basic catalyst to the selected polymer.

Since the average human osteon size is approximately 220 μm, the distribution function for the sizes of the pore-forming polymer microspheres will, preferably, have the mean value of 220 μm. Although high porosity enhances the growth of new bone into bone grafts, high porosity adversely affects important mechanical characteristics of the bone grafts. The volume fraction of polymer microspheres in the composite bone grafts usually are greater than 20%. Although at the volume fraction of 20%, the polymer particles are not completely interconnected, the dissolution of polymer particles at the surface will lead to surface pores for the deposition of new bone.

In order to maintain the mechanical properties of a composite bone graft and still enhance the attachment of new bone to the graft, a portion of polymer microspheres that degrade slowly to maintain mechanical integrity of the graft can be replaced with polymer microspheres that degrade rapidly [e.g., poly(sebacic acid anhydride)] or water soluble crystals that dissolve rapidly (e.g. crystals of salicylic acid or mannitol or aspirin). The addition of rapidly dissolving pore-forming particles will rapidly create some surface macropores that enhance the deposition of new bone. The replacement will be random or designed to have an enrichment of rapidly dissolving particles near the surface.

B. Moldable, Osteoconductive, Osteoinductive Biocompatible and Resorbable, Composite Bone Grafts

1. Composite Bone Grafts Consisting of Calcium Phosphate Cement and Growth-Factor Encapsulating Polymer Microspheres (or Combinations of Polymer Microspheres and Growth-Factor Encapsulating Polymer Microspheres)

In a typical example, Protein A (a representative bioactive agent model for bone growth factors) was encapsulated in PLGA microspheres by a double-emulsion technique. Each specimen of the composite containing the CPC and the Protein A-encapsulating PLGA microspheres was prepared by mixing 0.05 g of the encapsulating microspheres with 0.075 g of CPC on a glass plate and then mixing the admixture with 0.031 g of water to make a paste. The paste was placed into a stainless steel mold, 4 mm in diameter and 4.5 mm in height. Two 10 mm long steel inserts were placed sandwiching the specimen, and the mold was placed in a 37° C. humidity chamber. A load of 20 N was applied to the specimen for 24 h. The specimen was then removed and placed in a square fluorescence cell with a cross-sectional area of 1 cm². The cell was then filled with a known amount of 0.2 mol/L phosphate buffered saline solution (typically 4 g), sealed with a Teflon stopper, and stored in a 37° C. oven. The release of Protein A from the composite into the phosphate buffered saline solution was measured by fluorimetry as a function of time.

The release of Protein A was also measured for a composite that was the same as the aforementioned composite excepting that 65% of the Protein A-encapsulating PLGA microspheres were replaced with the PLGA microspheres containing no Protein A.

It was found that although the two composites contained different amounts of Protein A, the patterns for the release of Protein A from the two composites were similar. It was observed for both composites that although approximately half of the extractable Protein A was released in 170 h, the release of Protein A persisted for 840 h. Thus, composite bone grafts consisting of calcium phosphate cement and growth factor-encapsulating polymer microspheres (or combinations of polymer microspheres and growth factor-encapsulating polymer microspheres) are osteoinductive because they allow for controlled release of one or more bone growth factors.

2. Composite Bone Grafts Consisting of Calcium Phosphate Cement and Combinations of Polymer Microspheres and Polymer Microspheres Encapsulating Cells or Vectors for Gene Transfer

Instead of growth factor-encapsulating polymer microspheres that were described in the last section, the composite bone grafts may contain (in addition to pore-forming polymer microspheres or water-soluble particles) calcium-alginate microspheres or other polymer microspheres that encapsulate other bioactive agents, such as: 1) viral or non-viral vector or vectors for gene transfer (e.g., naked plasmid DNA encoding human parathyroid hormone [hPTH 1-34] or its complex with a cationic polymer) or 2) osteoblasts, or transfected cells producing bone morphogenetic proteins, or human bone marrow stromal cells, or pluripotent adult mesenchymal stem cells. For gene transfer applications or other considerations, encapsulating particles are designed to dissolve after the cement sets. For example, calcium-alginate microspheres may be mixed with particles of sodium citrate or sodium phosphate (or one of other chelating compounds for calcium ions) that dissolve calcium-alginate microspheres.

3. Composite Bone Grafts Consisting of Calcium Phosphate Cement, Biodegradable Microspheres and Growth-Factor Containing Liquid Component

For this release study, fluorescein-labeled Protein A was not encapsulated in the microspheres, because it is believed that it would be easier to add Protein A directly to the liquid component of the CPC. Four specimens were used to measure the change in the relative fluorescence intensity for fluorescein-labeled Protein A, released from the composite that consisted of the CPC and PLA [poly(lactic acid)] microspheres at the volume fraction of 0.6. The release pattern of fluorescein-labeled Protein A for this composite was similar to that for the composites containing Protein A-encapsulating PLGA microspheres. Although approximately half of the extractable Protein A was released in 140 h, the release of Protein A persisted for 640 h. Thus, composite bone grafts consisting of calcium phosphate cement, biodegradable microspheres and growth-factor containing liquid component are osteoinductive because they allow for controlled release of one or more bone growth factors.

4. Composite Bone Grafts Consisting of Calcium Phosphate Cement, Various Volume Fractions of Mannitol Crystals and Growth-Factor Containing Liquid Component

For this release study, fluorescein-labeled Protein A was added directly to the liquid component of the CPC. For each specimen, the fluorescence intensity of fluorescein-labeled Protein A released into the phosphate buffered saline solution was divided by the mass of the specimen to correct for the variation in mass. The corrected fluorescence intensity was then divided by the fluorescence intensity of the reference solution to yield the relative fluorescence intensity of the released Protein A. The changes in the relative fluorescence intensity for fluorescein-labeled Protein A, released from the composites that consisted of the CPC and mannitol crystals at volume fractions of 0%, 35% and 60%, respectively were observed. It was found that, up to 380 h, the amount of Protein A released from the composite increased with the increasing volume fraction of mannitol crystals. Thus, the release pattern of Protein A from the composite can be modulated through the control of the volume fraction of mannitol crystals. Similar results were obtained for the release of a bone growth factor (TGF-β1) with the use of mannitol crystals or salicylic-acid crystals as the pore-forming particles. Since the. rate for the formation of macropores depends on the dissolution rate of the pore-forming particles, the release pattern of a bone growth factor from the composite bone graft can be modulated through the control of the volume fraction and the dissolution rate of pore-forming particles.

5. Composite Bone Grafts Consisting of Calcium Phosphate Cement, Various Volume Fractions of Chondroitin Sulfate and Growth-Factor Containing Liguid Component

For this release study, fluorescein-labeled Protein A was added directly to the liquid component of the CPC. For each specimen, the fluorescence intensity of fluorescein-labeled Protein A released into the phosphate buffered saline solution was divided by the mass of the specimen to correct for the variation in mass. The corrected fluorescence intensity was then divided by the fluorescence intensity of the reference solution to yield the relative fluorescence intensity of the released Protein A. The changes in the relative fluorescence intensity for fluorescein-labeled Protein A, released from the composites that consisted of the CPC and chondroitin-sulfate at volume fractions of 0%, 30% and 40%, respectively were observed. It was found that, up to 350 h, the amount of Protein A released from the composite increased with the increasing volume fraction of chondroitin sulfate. Thus, the release pattern of Protein A from the composite can be modulated through the control of the volume fraction of chondroitin sulfate. Since the rate for the formation of macropores depends on the dissolution rate of the pore-forming water-soluble polymer, the release pattern of a bone growth factor from the composite bone graft can be modulated through the control of the volume fraction and the dissolution rate of the water-soluble polymer.

Calcium phosphate cement (CPC) can be improved by incorporating porogens to induce macropores and proteins to stimulate bone growth. Composite bone grafts made of CPC and polymer microspheres and those made of CPC and mannitol crystals both exhibit protein release. A paste was made from CPC powder (0.15 g equimolar mixture of tetracalcium phosphate and dicalcium phosphate), poly(d,l-lactide-co-glycolide) microspheres [0.1 g, 0.6 volume fraction, (0.17-0.36) mm dia.], and 0.062 g water. Discs (D=6.4 mm, H=4.4 mm) for diametral tensile strength (DTS) were prepared by applying a 2 kg load on the paste in a mold at 37° C. Discs (D=4 mm, H=4.5 mm) for the release of fluorescent Protein A (a model for growth factors) were similarly prepared, using poly(d,l-lactic acid) microspheres [PLA, (0.17-0.36) mm dia., 0.6 volume fraction] or mannitol crystals [(0.12-0.25) mm length; 0.0, 0.35 or 0.60 volume fraction) as porogens and a Protein A solution (1.3 mg Protein A per mL of 4 mol/L urea solution). Discs for DTS and Protein A release were stored at 37° C. in a saliva-like solution and a phosphate buffered saline solution, respectively. The initial and three-week DTS values for composite discs were approximately 6.4±0.9 MPa and approximately 3.5±0.3 MPa, comparable to 10.8 MPa for CPC discs. Half the extractable Protein A was released from CPC/PLA discs in 140 h. The release rate of Protein A from CPC/mannitol discs increased with mannitol volume fraction. With 0.6 volume fraction of microspheres, DTS values of composite grafts were similar to that of CPC. The release of proteins from composite grafts can be modulated by the volume fraction and the dissolution rate of porogens. As used throughout the description and claims, the term porogen includes material which create spherical and non-spherical voids, such as channels or other interconnected voids.

### EXAMPLE 2

A. Materials:

Poly(d,l-lactide-co-glycolide) (PLGA) (50:50) copolymer of average molecular mass of 91,200 g/mol, was obtained from Birmingham Polymers Inc., Birmingham, Ala. Poly(d,l-lactic acid) (PLA) of average molecular mass of 2,140 g/mol, was received from Boehringer Inglheim Chemicals, Inc., Petersburg, Va. Polyvinyl alcohol (PVA), mole fraction of 88% hydrolyzed and with molecular mass of 25,000 g/mol, was obtained from Polysciences Inc., Warrington, Pa. Protein A labeled with fluorescein isothiocyanate (FITC) was obtained from Sigma Chemical Co., St. Louis, Mo. Rubrene was obtained from K&K Laboratories, Plainview, N.Y. Dicalcium phosphate anhydrous (CaHPO₄) (DCPA) and CaCO₃ were obtained from J.T. Baker Chemical Co., Phillisburg, N.J. Tetracalcium Phosphate [Ca₄(PO₄)O (TTCP)] was prepared as described in “Formation of macropores in calcium phosphate cement implants”, J Mater Sci: Mater Med 2000; 49: 183-191, herein incorporated by reference in its entirety. Calcium phosphate cement (CPC) powder, consisting of an equimolar mixture of TTCP (mass fraction of 72.9%) and DCPA (mass fraction of 27.1%) was prepared by thoroughly mixing the ground TTCP and DCPA in a blender. 4-(2-hydroxyethyl)-1-piperazineethanesulfonic acid (HEPES) was obtained from United States Biochemical Corporation, Cleveland, Ohio. CaCl₂, K₂HPO₄, KCl and NaN₃ of highest purity were obtained from Sigma Chemical Co., St. Louis, Mo. Laboratory grade thymol was obtained from Fisher Scientific, Fair Lawn, N.J. Gelatin (75 bloom, Type B, from bovine skin) was obtained from Sigma Chemical Co., St. Louis, Mo. Guanidine hydrochloride was obtained from Aldrich Chemical Co., Milwaukee, Wis. Dichloromethane was obtained from J.T. Baker Chemical Co., Phillisburg, N.J.

B. Preparation of Microspheres

PLGA microspheres were prepared by adding a solution of 4 g of PLGA dissolved in 20 mL of dichloromethane, all at once, to 1.6 L of a PVA solution (mass fraction of 1% in distilled water) in a 2 L beaker. The solution was stirred at 375 revolutions per minute using a mechanical stirrer at room temperature in a well-ventilated hood. A motorized stirrer (Caframo, Model BBC 3030, available from Fisher Scientific) equipped with a stainless-steel cross-blade impeller was used. The length of each blade (i.e., one half the diameter of the propeller) was 2.5 cm. The width and the thickness of each blade were 1.2 cm and 0.5 mm, respectively. After 4.5 h of stirring, the contents were allowed to stand for 30 min. Most of the liquid was decanted and the solid was washed four times with 250 mL of distilled water and then transferred onto a Whatman 1 filter paper and washed with 500 mL of water. Microspheres were transferred into a 40 mL vial with a diameter of about 3.8 cm. Distilled water (7 mL) was added to the bottle and the contents were frozen using dry ice-acetone while rotating the vial to create a large surface area for the frozen matrix. The bottle was then attached to a lyophilizer until a constant weight of the bottle was recorded. In this way 3.73 g of free-flowing microspheres were obtained.

To obtain PLA microspheres, 16 g of PVA was dissolved in 1.6 L of warn distilled water in a 2 L beaker and brought to room temperature. To this was added 8 g of PLA dissolved in 20 mL of dichloromethane while stirring the mixture at 275 revolutions per minute. After 4 h, stirring was stopped and the contents were left standing for 30 min. Most of the liquid was decanted and the solid was washed four times with 250 mL of distilled water and transferred into a 40 mL vial with a diameter of about 3.8 cm. Distilled water (10 mL) was added to the bottle and contents were frozen using dry ice-acetone while rotating the vial to create a large surface area for the frozen matrix. It was then attached to a lyophilizer until a constant weight of the bottle was recorded. In this way 6.72 g of free-flowing microspheres were obtained.

C. Fractionation of Microspheres

18.8 g of PLGA microspheres were transferred onto a size #45 sieve that was placed on a size #80 sieve above a collector plate. The top sieve was covered and then the assembly was placed on a mechanical shaker and shaken for 1.5 h. The size #45 sieve and the size #80 sieve used in the fractionation should allow particles between 170 μm and 360 μm to be collected on the size #80 sieve. Microspheres (11.3 g) remaining on the size #80 sieve were transferred into a vial and stored in a refrigerator until further use. The same procedure was used for the fractionation of PLA microspheres.

D. Preparation of PLGA/CPC Composite for Measurements of Diametral Tensile Strength (DTS) and Mass Loss

The volume fraction of the PLGA microspheres in the composite was 0.6. Each specimen of the PLGA/CPC composite was prepared by mixing 0.1 g of PLGA microspheres with 0.15 g of CPC on a glass plate and then mixing the admixture with 0.062 g of water to make a paste. The paste was placed into a stainless steel mold, 6.4 mm in diameter and 4.4 mm in height. Two 10 mm long steel inserts were placed sandwiching the specimen, and the mold was placed in a 37° C. humidity chamber. A load of 20 N was applied to the specimen for 24 h. The specimen was then removed and used for the DTS and the mass loss measurements.

E. Measurement of Diametral Strength (DTS) of PLGA/CPC Composite

To prepare a saliva-like solution, 0.8 mL of 3 mol/L CaCl₂, 23.8 g of HEPES, 4.47 g of KCl and 1.6 mL of 0.9 mol/L KH₂PO₄ were mixed in a 2 L beaker and 1800 mL of distilled water was added to the admixture. After mixing, the pH of the solution was adjusted to 7.0 using a KOH solution. The solution was transferred to a volumetric flask and diluted with distilled water to a final volume of 2 L. Three crystals of thymol were added as an antibacterial to the saliva-like solution.

Each specimen for DTS measurement was separately stored in 50 mL of the saliva-like solution at 37° C. The saliva-like solution was changed every week. Once a week for a period of six weeks, a set of five specimens was removed. After determining the diameter and the thickness of each specimen, the DTS of each specimen, which remained wet, was measured using an Instron 5500R. The value of the DTS was determined using the relation DTS=2F/(3.14 d×h) where F is the maximum load, d is the diameter of the specimen, and h is the thickness of the specimen.

F. Measurement of Mass Loss for PLGA/CPC Composite

Three specimens of the PLGA/CPC composite were prepared and freeze-dried to constant weights. They were then stored in separate bottles containing 50 mL of the saliva-like solution at 37° C. Specimens were periodically removed for measuring their masses after freeze-drying, and then replaced in the saliva-like solution at 37° C. The saliva-like solution was changed every week.

G. Scanning Electron Microscopy

Scanning electron microscopy (SEM) was used to examine the morphology of the PLGA/CPC composite after drying a specimen. The specimen was mounted on an aluminum stub and gold coated using a sputter coater set at 20 mA for a total time of 180 s. The specimen was then observed with a Jeol JSM 5300 SEM operated at 5 kV.

H. X-Ray Diffraction

X-ray diffraction measurements on specimens of the PLGA/CPC composite were performed with a Rigaku (Danvers, Mass.) instrument to determine the degree of hydroxyapatite formation. The estimated standard uncertainty of the 2θ measurement was 0.01° and the minimum mass fraction of a crystal phase to be detected by the instrument was about 3%.

I. Encapsulation of Protein A in PLGA Microspheres

2.2 mg of Protein A, labeled with fluorescein isothiocyanate, was added to a 4 mol/L solution containing 15.3 g of guanidine hydrochloride in 40 mL of distilled water. To 10 mL of this solution was added 0.81 g of bovine gelatin (75 bloom). Separately, 1 g of PLGA was dissolved in 15 mL of dichloromethane. The solution containing Protein A, gelatin and guanidine hydrochloride was added to the methylene-chloride solution of PLGA, and shaken to create an emulsion. The emulsified solution was added to a 2 L beaker containing 1.8 L of an aqueous gelatin solution (mass fraction of 1%) and stirred at 375 revolutions per minute for 4 h at room temperature. Thirty minutes after the end of stirring, the microspheres were separated from the liquid, washed with distilled water, and freeze-dried for three days to yield 1.44 g of colorless microspheres, mostly between 200 to 300 μm in diameter.

J. Release of Protein A from Composite of CPC and Protein A-Encapsulating PLGA Microspheres

Each specimen of the composite containing the CPC and the Protein A-encapsulating PLGA microspheres was prepared by mixing 0.05 g of the encapsulating microspheres with 0.075 g of CPC on a glass plate and then mixing the admixture with 0.031 g of water to make a paste. The paste was placed into a stainless steel mold, 4 mm in diameter and 4.5 mm in height. Two 10 mm long steel inserts were placed sandwiching the specimen, and the mold was placed in a 37° C. humidity chamber. A load of 20 N was applied to the specimen for 24 h. The specimen was then removed and placed in a square fluorescence cell with a cross-sectional area of 1 cm². The cell was then filled with a known amount of 0.2 mol/L phosphate buffered saline solution (typically 4 g), sealed with a Teflon stopper, and stored in a 37° C. oven. The release of Protein A from the composite into the phosphate buffered saline solution was measured by fluorimetry as a function of time.

K. Release of Protein A from Composite Consisting of CPC, PLGA Microspheres and Protein A-Encapsulating PLGA Microspheres

The mixed microspheres were prepared by mixing 35% mass fraction of the PLGA microspheres with 65% mass fraction of the Protein A-encapsulating PLGA microspheres. Each specimen of the composite consisting of the CPC, the PLGA microspheres and the Protein A-encapsulating PLGA microspheres was prepared by mixing 0.05 g of the mixed microspheres with 0.075 g of CPC on a glass plate and then mixing the admixture with 0.031 g of water to make a paste. In the manner described in the last paragraph for the release of Protein A from the composite of CPC and Protein A-encapsulating PLGA microspheres, the composite was prepared from the paste, and the release of Protein A from the composite into the phosphate buffered saline solution was measured by fluorimetry as a function of time.

L. Release of Protein A from Composite of CPC and PLA Microspheres

Each specimen of the composite containing Protein A, the CPC, and PLA microspheres was prepared by mixing 0.05 g of PLA microspheres with 0.075 g of CPC on a glass plate and then mixing the admixture with 0.031 g of a solution of Protein A in 4 mol/L urea solution (1.3 mg of Protein A per mL of the urea solution) to make a paste. In the manner described for the release of Protein A from the composite of CPC and Protein A-encapsulating PLGA microspheres, the composite was prepared from the paste, and the release of Protein A from the composite into the phosphate buffered saline solution was measured by fluorimetry as a function of time.

M. Release of Protein A from Composite of CPC and Mannitol Crystals

Mannitol crystals were recrystallized, ground, and sieved through 250 μm and 125 μm sieves. An admixture was prepared by mixing the CPC with sieved mannitol crystals at the volume fraction of 35% or 60%. Each specimen of the composite containing Protein A, the CPC, and mannitol crystals was prepared by mixing 0.125 g of the admixture with 0.031 g of a solution of Protein A in 4 mol/L urea solution (1.3 mg of Protein A per mL of the urea solution) to make a paste. In the manner described for the release of Protein A from the composite of CPC and Protein A-encapsulating PLGA microspheres, the composite was prepared from the paste, and the release of Protein A from the composite into the phosphate buffered saline solution was measured by fluorimetry as a function of time.

N. Fluorescence Measurement

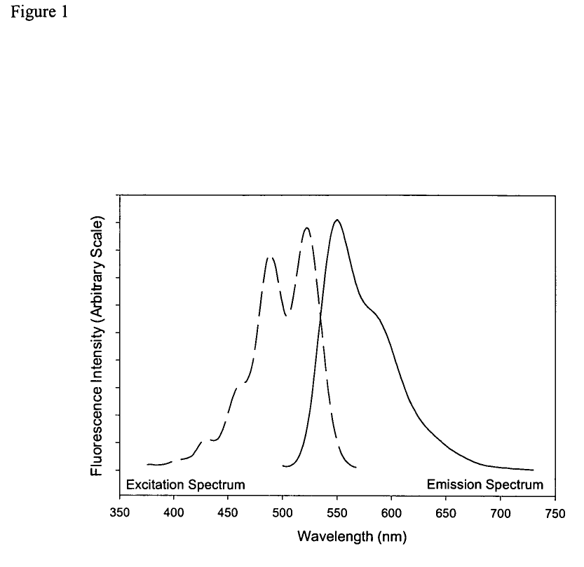

The fluorescence intensity of fluorescein-labeled Protein A in the phosphate buffered saline solution was measured at 22° C.±1° C. using a spectrofluorometer (Fluorolog 2 series, SPEX Industries, Inc., Metuchen, N.J.). The intensity was measured at an excitation wavelength of 480 nm and an emission wavelength of 540 nm, using the front-surface-illumination mode. The widths of the slits were 2.5 mm at all gates and a high pass filter of 495 nm was used at the entrance to the emission monochromator. A deoxygenated rubrene solution in ethyl acetate (2.19×10⁻⁵ mol/L), sealed in a quartz cuvette under vacuum, was used as a reference solution in order to correct for potential instrumental instability. The optical density of the reference solution was 0.031. FIG. 1 gives the excitation spectrum (measured at the emission wavelength at 670 nm) and the emission spectrum (measured at the excitation wavelength at 475 nm) for the reference solution. The fluorescence intensity of the fluorescein-labeled Protein A in the phosphate buffered saline solution and the fluorescence intensity of the reference solution were both measured at the excitation and emission wavelengths of 480 nm and 540 nm, respectively. The ratio of the two intensities was proportional to the concentration of Protein A, since the concentration was kept low enough to avoid the inner filter effect.

Results and Discussion

A. Characterization of Microspheres and PLGA/CPC Composite

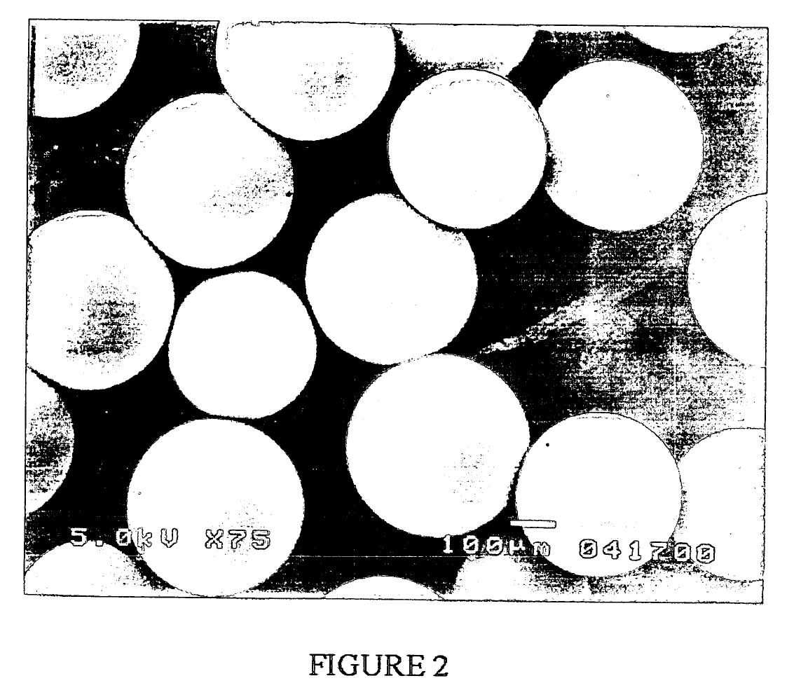

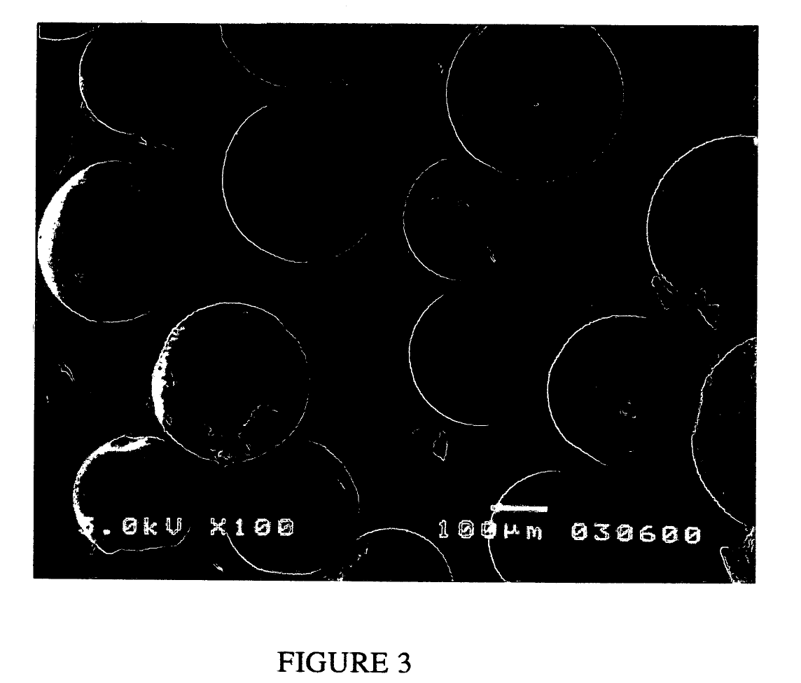

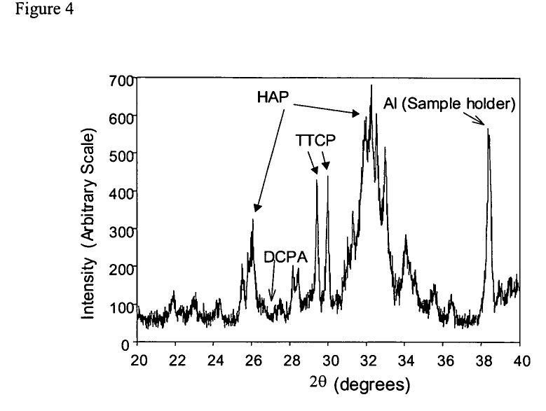

FIG. 2 and FIG. 3 show the SEM (scanning electron microscopy) micrographs of the fractionated PLGA microspheres and PLA microspheres, respectively. Each micrograph shows that the microspheres were not agglomerated and were between 170 μm and 360 μm in diameter. FIG. 4 gives the X-ray diffraction pattern for a specimen of the PLGA/CPC composite. The pattern shows that DCPA reacted completely while a small amount of TTCP remained, indicating the formation of calcium-deficient hydroxyapatite (HAP) in the composite specimen. An optical micrograph for the Protein A-encapsulating microspheres showed the sizes of the microspheres to be between 200 μm and 300 μm.

B. Degradation of PLGA in PLGA/CPC Composite

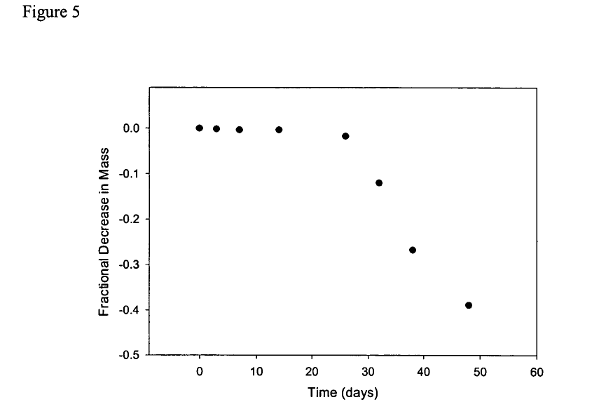

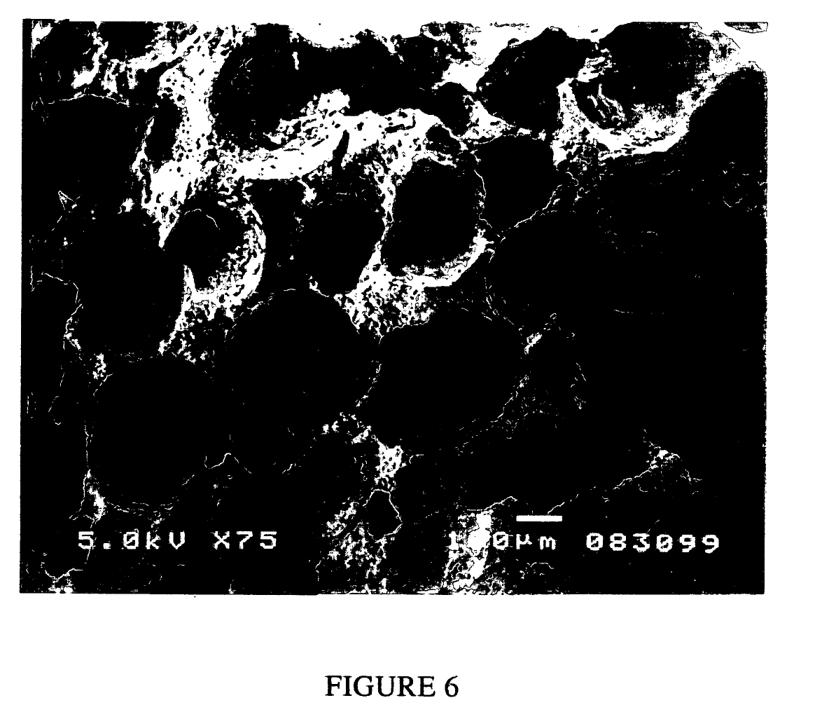

The degradation of PLGA in the PLGA/CPC composite was investigated by measuring, over a period of seven weeks, the decreases in mass for three freeze-dried composite specimens that were stored in different bottles containing the saliva-like solution at 37° C. FIG. 5 shows the average fractional decrease in mass as a function of time. It is worth noting that the mass did not decrease rapidly until after 26 days. The standard deviations of the values for the fractional decrease in mass were 4.0%, 1.2% and 1.4% on 32nd, 38th and 48th day, respectively. Swollen masses of PLGA, which was partially degraded and highly hydrated, exuded from the pores of the composite specimens after 21 days. After seven weeks, the mass of each composite specimen after freeze-drying was nearly the same as that of the CPC powder, suggesting a nearly complete dissolution of PLGA. Pores can be seen in the SEM micrograph (FIG. 6) of a small fragment from a composite specimen that was removed from the solution after seven weeks, indicating the complete dissolution of PLGA microspheres.

C. Diametral Tensile Strength (DTS) of PLGA/CPC Composite.

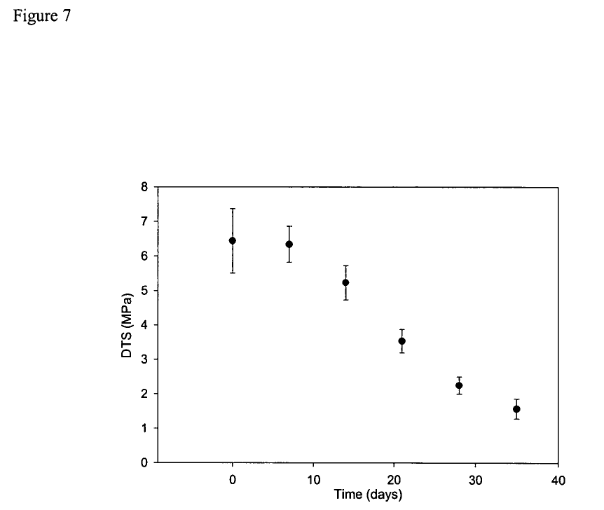

Specimens for DTS measurements were stored at 37° C. in 50 mL of the saliva-like solution that was changed once every week. Once a week for a period of six weeks, DTS measurements were carried out on a set of five specimens while they were still wet. FIG. 7 gives the average value of the DTS as a function of time. It is worth noting that even though FIG. 5 shows very little decrease in the mass of the PLGA/CPC composite during the first four weeks of the experiment, FIG. 7 shows a steady decrease in the value of DTS during this period. There was little decrease in the mass of the composite during this period, probably because the degradation reaction of PLGA with water (permeating into the composite through the micropores of CPC) was not sufficient to produce PLGA molecules that were low enough in molecular mass to diffuse out of the composite. However, the reduction in the average molecular mass of PLGA, together with the hydration-induced softening of PLGA microspheres, caused the decrease in DTS shown in FIG. 7. The initial value of 6.4 MPa for the DTS of the PLGA/CPC composite (containing 60% by volume of PLGA microspheres) was higher than the value of 3.5±0.4 MPa (n=3) for the CPC disk with 29% by volume of mannitol-induced macropores. After 21 days of immersion in the saliva-like solution, the PLGA/CPC composite still retained the DTS value of 3.5 MPa, the same as the value for the CPC disk with 29% by volume of mannitol-induced macropores.

D. Release of Protein A from Composite of CPC and Protein A-Encapsulating PLGA Microspheres

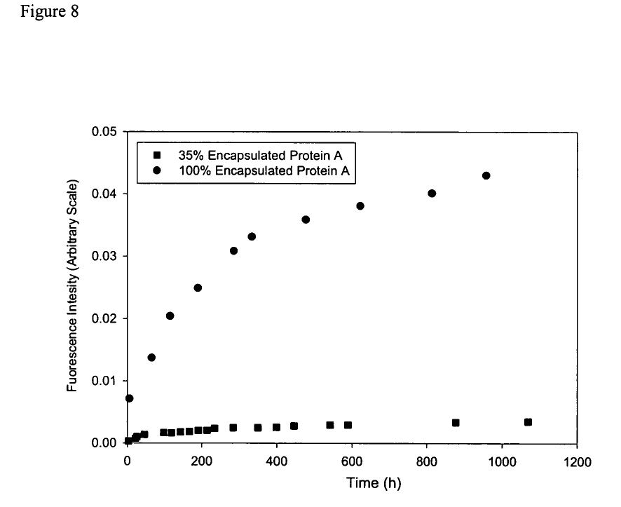

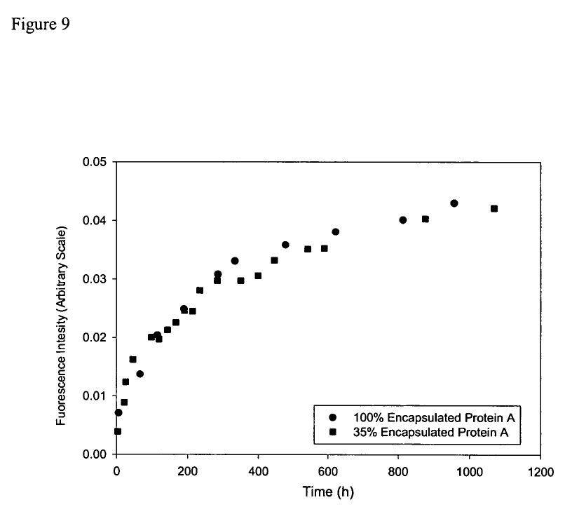

The release of Protein A, which had been previously used as a model for growth factors, was measured to lay the groundwork for the more relevant studies on the release of rhTGF-β1 from the composite bone grafts. In FIG. 8 the upper plot gives the change in the relative fluorescence intensity for fluorescein-labeled Protein A, released from the composite that consisted of the CPC and Protein A-encapsulating PLGA microspheres at the volume fraction of 0.6. The lower plot gives the change in the relative fluorescence intensity for fluorescein-labeled Protein A, released from the same composite as the aforementioned composite excepting that 65% of the Protein A-encapsulating PLGA microspheres were replaced with the PLGA microspheres. It is worth noting that although the two composites contained different amounts of Protein A, the patterns for the release of Protein A from the two composites were similar. The similarity of the release patterns is more readily seen when the relative fluorescence intensity for the lower plot is multiplied by a factor of 12, as shown in FIG. 9. In FIG. 9, it is shown for both composites that although approximately half of the extractable Protein A was released in 170 h, the release of Protein A persisted for 840 h.

E. Release of Protein A from Composite of CPC and PLA Microspheres

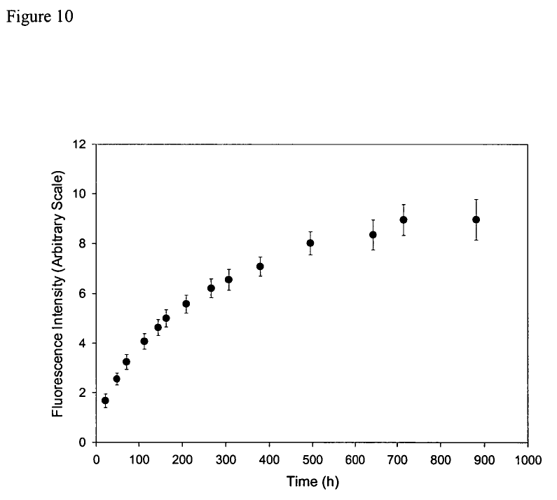

For this release study, fluorescein-labeled Protein A was not encapsulated in the microspheres, since we realized that it would be easier to add Protein A directly to the liquid component of the CPC. Four specimens were used to measure the change in the relative fluorescence intensity for fluorescein-labeled Protein A, released from the composite that consisted of the CPC and PLA microspheres at the volume fraction of 0.6. For each specimen, the fluorescence intensity of fluorescein-labeled Protein A released into the phosphate buffered saline solution was divided by the mass of the specimen to correct for the variation in mass. The corrected fluorescence intensity was then divided by the fluorescence intensity of the reference solution to yield the relative fluorescence intensity of the released Protein A. FIG. 10 gives the change in the average of the relative fluorescence intensities for fluorescein-labeled Protein A released from the specimens. The release pattern was similar to that shown in FIG. 9 for the composites containing Protein A-encapsulating PLGA microspheres. Although approximately half of the extractable Protein A was released in 140 h, the release of Protein A persisted for 640 h.

F. Release of Protein A from Composite of CPC and Mannitol Crystals

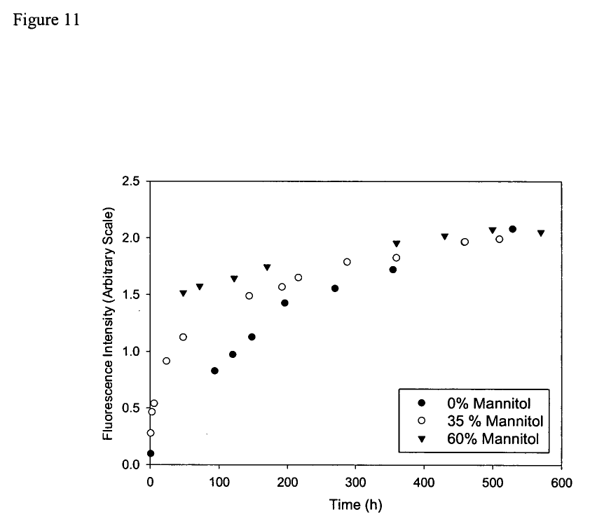

For this release study, fluorescein-labeled Protein A was added directly to the liquid component of the CPC. For each specimen, the fluorescence intensity of fluorescein-labeled Protein A released into the phosphate buffered saline solution was divided by the mass of the specimen to correct for the variation in mass. The corrected fluorescence intensity was then divided by the fluorescence intensity of the reference solution to yield the relative fluorescence intensity of the released Protein A. FIG. 11 gives the changes in the relative fluorescence intensity for fluorescein-labeled Protein A, released from the composites that consisted of the CPC and mannitol crystals at volume fractions of 0%, 35% and 60%, respectively. It is worth noting that, up to 380 h, the amount of Protein A released from the composite increased with the increasing volume fraction of mannitol crystals. Thus, the release pattern of Protein A from the composite can be modulated through the control of the volume fraction of mannitol crystals.

Thus, the degradation kinetics and mechanical properties of a novel moldable, resorbable, composite bone graft consisting of calcium phosphate cement (CPC) and poly(d,l-lactide-co-glycolide) [PLGA] microspheres has been shown, along with several methods for controlling the release of bioactive molecules from the moldable composite bone graft through inclusion of biodegradable particles. In contrast to prior compositions, the composite bone grafts of the invention are the first ‘polymer-in-mineral’ formulations, which are made of PLGA or PLA microspheres and a calcium phosphate matrix (CPC).

The mass of the PLGA/CPC composite (containing 60% by volume of PLGA microspheres) did not decrease rapidly until after four weeks. The mass loss due to the degradation and the dissolution of PLGA microspheres was complete in seven weeks. The initial value of the diametral tensile strength (DTS) for the PLGA/CPC composite (containing 60% by volume of PLGA microspheres) was higher than the value of 3.5±0.4 MPa (n=3) for the CPC disk with 29% by volume of mannitol-induced macropores.

Two types of PLGA/CPC composite bone grafts that contained 60% by volume of PLGA microspheres were used to measure the release of Protein A, a model for growth factors. In the first type, all the microspheres contained Protein A, while in the second type, 35% of the microspheres contained the protein and 65% did not. For both composites, approximately half of the extractable Protein A was released in 170 h, although the release of Protein A persisted for 840 h. In another method for incorporating Protein A into the composite bone grafts, Protein A was directly added to the liquid component of the CPC. For the composite that consisted of the CPC and PLA microspheres at the volume fraction of 0.6, approximately half of the extractable Protein A was released in 140 h, although the release of Protein A persisted for 640 h. The differences in the release pattern of Protein A from the composites that contained 0%, 35% and 60% by volume, respectively, of mannitol crystals demonstrated that the release pattern of Protein A can be modulated through the control of the volume fraction of mannitol crystals. Since at the volume fraction of 60%, the release of Protein A was faster with faster-dissolving manitol crystals as porogens than with PLA microspheres as porogens, the release pattern of Protein A can also be modulated by controlling the dissolution rate of porogens.

Thus, the present inventor has demonstrated several methods to modulate the kinetics of growth-factor release from the composite bone graft. For the first method, the growth factor is dissolved in the liquid component and no porogens are dispersed in the cement powder. For the second method, the growth factor is dissolved in the liquid component, and degradable polymeric microspheres (or soluble crystals such as mannitol crystals) are dispersed as porogens in the cement powder. Thus, for a fixed volume fraction of porogens, the growth-factor release can be increased by increasing the rate of dissolution of porogens that results in a faster formation of macropores. Furthermore, for a given type of porogens in the second method, the growth-factor release can be increased by increasing the volume fraction of porogens that results in a larger volume fraction of macropores. For the third method, the growth factor is not dissolved in the liquid component, but degradable polymeric microspheres that encapsulate the growth factor are dispersed as porogens in the cement powder. As was described for the second method, the growth-factor release can be modulated by controlling the degradation rate and the volume fraction of the encapsulating microspheres. Finally, in the fourth method, some fraction of the porogens for the second method is replaced with degradable polymeric microspheres that encapsulate the growth factor or some other bioactive ingredient.

It should be apparent that embodiments other than those specifically described above may come within the spirit and scope of the present invention. Hence, the present invention is not limited by the above description, but rather is defined by the claims appended hereto.

We claim:

1. A moldable, resorbable composite bone graft material comprising:
calcium phosphate cement (CPC), an aqueous medium, and
biodegradable porogen, optionally comprising polymer microspheres, and/or water-soluble particles,
wherein the bone graft formed from the bone graft material is osteoconductive and osteoinductive.

2. The bone graft material of claim 1, wherein the CPC consists of an equimolar mixture of tetracalcium phosphate (TTCP) powder and dicalcium phosphate anhydrous (DCPA) powder.

3. The bone graft material of claim 1, wherein the distribution function for the sizes of the microspheres has a mean value of 220 μm.

4. The bone graft material of claim 1, wherein the microspheres have diameters between approximately (80 and 360) μm.

5. The bone graft material of claim 1, wherein the volume fraction of microspheres is at least 20%.

6. The bone graft material of claim 1, wherein the biodegradable porogen comprises at least one selected from the group consisting of mannitol crystals, poly(L-lactide), poly(glycolide), poly(D,L-lactide), poly(ε-caprolactone), poly(carbonates) and poly(orthoesters), as well as poly(anhydrides), poly(D,L-lactide-co-L-lactides), poly(D,L-lactide-co-glycolides) and poly(L-lactide-co-glycolides), poly(sebacic acid anhydride), salicylic acid crystals, chondroitin sulfate, and aspirin.

7. The bone graft material of claim 6, wherein the volume fraction of mannitol crystals is less than approximately 0.6.

8. The bone graft material of claim 1, further comprising a bioactive agent.

9. The bone graft material of claim 8, wherein the bioactive agent comprises at least one selected from the group consisting of bone growth factors, vectors for gene transfer, and human cells.

10. The bone graft material of claim 9, wherein the bone growth factor comprises at least one selected from the group consisting of a bone morphogenetic protein and TGF-β1.

11. The bone graft material of claim 9, wherein the bioactive agent is encapsulated in at least some of the biodegradable porogen.

12. The bone graft material of claim 9, wherein the bioactive agent is encapsulated in all of the biodegradable porogen.

13. The bone graft material of claim 9, wherein the human cells comprise at least one selected from the group consisting of osteoblasts, transfected cells producing bone morphogenetic proteins, human bone marrow stromal cells, pluripotent adult mesenchymal stem cells.

14. The bone graft material of claim 9, wherein the vectors comprise a naked plasmid DNA encoding human parathyroid hormone [hPTH 1-34] or its complex with a cationic polymer.

15. The bone graft material of claim 1, wherein the mechanical strength of the bone graft formed from the bone graft material is at least approximately 6 MPa in diametral tensile strength.

16. The bone graft material of claim 1, wherein biodegradable porogens dissolve at a rate less than a rate at which the polymer microspheres dissolve.

17. A method for producing an osteoinductive and osteoconductive bone graft comprising the steps of:
(a) providing calcium phosphate cement (CPC),
(b) providing biodegradable porogens, and
(c) combining the CPC, an aqueous medium, and the biodegradable porogens to form a bone graft material;
(d) applying the bone graft material in vivo.

18. The method of claim 17, wherein step (d) comprises at least one step selected from the group consisting of injecting and molding.

19. The method of claim 17, further comprising prior to step (d), combining a bioactive agent in the bone graft material.

20. The method of claim 16, wherein step (c) comprises encapsulating at least a portion of the bioactive agent into the biodegradable porogen.

21. The method of claim 19, wherein step (c) comprises encapsulating all of the bioactive agent into the biodegradable porogen.

22. The method of claim 17, further comprising the step of:
(e) allowing, in vivo, osteoblasts to invade the voids formed by the degradation of the biodegradable porogen.

23. The method of claim 17, further comprising the steps of:
(e) providing at least one dissolvable material selected from the group consisting of polymer microspheres and water soluble particles, the dissolvable material having a known dissolution rate;
(e) combining dissolvable material with the product of step (c);
wherein the dissolution rate of the dissolvable material is greater than a dissolution rate of the porogens.
